(19) United States
(12) Patent Application Publication
(10) Pub. No.: US 2022/0039810 A1
(43) Pub. Date: Feb. 10, 2022

(54) METATARSAL ALIGNMENT APPARATUS

(71) Applicant: In2Bones USA, LLC, Memphis, TN (US)

(72) Inventors: Carlos E. Gil, Memphis, TN (US); Robert M. Crews, Memphis, TN (US); Rebecca Hawkins Wahl, Escondido, CA (US)

(73) Assignee: In2Bones USA, LLC, Memphis, TN (US)

(21) Appl. No.: 17/398,928

(22) Filed: Aug. 10, 2021

Related U.S. Application Data
(60) Provisional application No. 63/063,889, filed on Aug. 10, 2020.

(51) Int. Cl. A61B 17/17; A61B 17/68

(52) U.S. Cl. CPC A61B 17/1775; A61B 2017/00367; A61B 17/68

(57) ABSTRACT

A metatarsal alignment apparatus and methods are provided for orienting and maintaining a 1st metatarsal bone in a natural anatomical position during surgical procedures for treating a hallux valgus deformity by way of arthrodesis of the 1^{st} metatarsocuneiform joint. The metatarsal alignment apparatus includes a cuneiform block for pinning a cuneiform bone and a distal metatarsal block for pinning a 1^{st} metatarsal bone. The cuneiform block is threadably engaged with a shaft for moving the cuneiform block longitudinally with respect to the distal metatarsal block. The distal metatarsal block is adjustable within a distal frame to facilitate moving transversely and rotating the 1^{st} metatarsal bone with respect to the cuneiform bone. The distal metatarsal block may be fixated by way of a lock screw to fixate the orientation of the 1^{st} metatarsal bone with respect to the cuneiform bone during preparing the 1^{st} metatarsocuneiform joint for arthrodesis.

### PRIORITY

This application claims the benefit of and priority to U.S. Provisional Application, entitled “Metatarsal Alignment Apparatus,” filed on Aug. 10, 2020 and having application Ser. No. 63/063,889, the entirety of said application being incorporated herein by reference.

### FIELD

Embodiments of the present disclosure generally relate to the field of securing bones together. More specifically, embodiments of the disclosure relate to an apparatus and methods for maintaining a 1^{st} metatarsal bone in a natural anatomical position during surgical procedures.

### BACKGROUND

Hallux valgus is a progressive foot deformity wherein the distal region of the big toe (i.e., the “hallux”) deviates in a lateral direction. Such a deformity can be caused by wearing pointed shoes with a narrow toe box. For example, when wearing high heel shoes, the foot is forced into the front of the shoe. The narrow front of the shoe forces the distal hallux in the lateral direction, toward the other toes, while a distal portion of the 1^{st} metatarsal head is forced in a medial direction. Forcing the distal metatarsal head in the medial direction pushes it outward and against an edge of the shoe. The irritation caused by pressing the metatarsal head against the shoe often causes an enlarged and thickened callus, or a bunion, to form.

A hallux valgus deformity may have significant ramifications for soft tissue problems in other areas, such as pain and functional deficit. For example, a hallux valgus deformity can give rise to an impaired gait characterized by lateral and posterior weight shift, late heel rise, decreased single-limb balance, pronation deformity, and the like. When the hallux is deviating away from its normal position, it does not have the mechanical ability to perform these tasks correctly. For example, if the hallux is not preventing overpronation, a number of other problems may develop, including plantar fasciitis, shin splints, or other ankle or knee pathologies.

Given that hallux valgus is relatively prevalent in the general population, there is an ongoing need for the development of foot treatment capabilities such as that related to, for example, treating hallux valgus deformities. Provided herein are embodiments and methods for maintaining a 1^{st} metatarsal bone in a natural anatomical position during surgical procedures for treating a hallux valgus deformity by way of arthrodesis of the 1^{st} metatarsocuneiform joint.

### SUMMARY

A metatarsal alignment apparatus and methods are provided for orienting and maintaining a 1^{st} metatarsal bone in a natural anatomical position during surgical procedures for treating a hallux valgus deformity by way of arthrodesis of the 1^{st} metatarsocuneiform joint. The metatarsal alignment apparatus includes a cuneiform block for pinning a cuneiform bone and a distal metatarsal block for pinning a 1^{st} metatarsal bone. The cuneiform block is threadably engaged with a shaft disposed within the proximal frame such that turning the shaft moves the cuneiform block longitudinally with respect to the distal metatarsal block. The distal metatarsal block is adjustable within a distal frame and configured to facilitate moving and rotating the 1^{st} metatarsal bone with respect to the cuneiform bone. The distal metatarsal block and the distal frame facilitate moving the 1^{st} metatarsal bone along a transverse direction with respect to the cuneiform bone. The distal metatarsal block may be fixated by way of a lock screw to fixate the orientation of the 1^{st} metatarsal bone with respect to the cuneiform bone during preparing the 1^{st} metatarsocuneiform joint for arthrodesis.

In an exemplary embodiment, a metatarsal alignment apparatus comprises: a cuneiform block slidably disposed within a proximal frame; a distal metatarsal block adjustably disposed within a distal frame; and a threaded shaft for moving the cuneiform block relative to the distal metatarsal block.

In another exemplary embodiment, the distal frame is configured for use on either a left foot or a right foot of a patient. In another exemplary embodiment, the threaded shaft is longitudinally disposed within the proximal frame and threadably engaged with the cuneiform block. In another exemplary embodiment, the proximal frame is configured to guide the cuneiform block in a longitudinal direction as the threaded shaft is turned. In another exemplary embodiment, the threaded shaft includes a shaped opening configured to be engaged with a suitable rotary tool for the purpose of turning the threaded shaft to move a cuneiform bone.

In another exemplary embodiment, the cuneiform block includes vertical holes and angled holes for pinning the cuneiform block to the cuneiform bone. In another exemplary embodiment, the distal metatarsal block includes a cannulation configured for pinning the distal metatarsal block to a 1^{st} metatarsal bone. In another exemplary embodiment, the distal metatarsal block includes a shaped opening for receiving a tool whereby the distal metatarsal block may be moved along slots disposed in the distal frame. In another exemplary embodiment, the slots are configured to rotate the distal metatarsal block with respect to the cuneiform block. In another exemplary embodiment, a lock screw is configured to be tightened to fixate the orientation of the distal metatarsal block with respect to the distal frame.

In another exemplary embodiment, the threaded shaft is longitudinally disposed within the proximal frame and threadably engaged with both the cuneiform block and a metatarsal block. In another exemplary embodiment, the metatarsal block includes vertical holes and angled holes for pinning the metatarsal block to a 1^{st} metatarsal bone. In another exemplary embodiment, the proximal frame is configured to guide the cuneiform block and the metatarsal block along the longitudinal direction as the threaded shaft is turned. In another exemplary embodiment, the threaded shaft engages the cuneiform block with left-hand threads and engages the metatarsal block with right-hand threads, such that turning the threaded shaft causes the cuneiform block and the metatarsal block to move in opposite directions.

In another exemplary embodiment, the distal metatarsal block includes vertical holes configured for pinning the distal metatarsal block to a 1^{st} metatarsal bone of the patient. In another exemplary embodiment, the distal metatarsal block includes a shaped opening configured for receiving a suitable tool whereby the block may be moved along slots disposed in the distal frame. In another exemplary embodiment, the slots are configured to allow the distal metatarsal block to move along a transverse direction with respect to the proximal frame and to rotate about its axis with respect to the cuneiform block. In another exemplary embodiment, a distal head lock screw is configured to be tightened to fixate the orientation of the distal metatarsal block with respect to the distal frame.

In another exemplary embodiment, the distal frame is configured to be rotated with respect to the proximal frame and to be fixated to the proximal frame by way of a locking knob. In another exemplary embodiment, the distal frame is configured to be used to adjust a rotation angle of the 1^{st} metatarsal bone with respect to the cuneiform bone.

These and other features of the concepts provided herein may be better understood with reference to the drawings, description, and appended claims.

### BRIEF DESCRIPTION OF THE DRAWINGS

The drawings refer to embodiments of the present disclosure in which:

FIG. 1 illustrates an exemplary embodiment of a metatarsal alignment apparatus configured for use on either a left foot or a right foot of a patient, according to the present disclosure;

FIG. 2 illustrates an exemplary embodiment of a left frame and a right frame respectively configured for a left-foot metatarsal alignment apparatus and a right-foot metatarsal alignment apparatus in accordance with the present disclosure;

FIG. 3 illustrates an exemplary embodiment of a metatarsal alignment apparatus configured for use on a left foot of a patient, according to the present disclosure;

FIG. 4 illustrates an exemplary embodiment of a metatarsal alignment apparatus configured for use on a left foot of a patient, in accordance with the present disclosure; and

FIG. 5 illustrates an exemplary embodiment of a metatarsal alignment apparatus comprising an independent distal frame, according to the present disclosure.

While the present disclosure is subject to various modifications and alternative forms, specific embodiments thereof have been shown by way of example in the drawings and will herein be described in detail. The invention should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the invention disclosed herein may be practiced without these specific details. In other instances, specific numeric references such as “first portion,” may be made. However, the specific numeric reference should not be interpreted as a literal sequential order but rather interpreted that the “first portion” is different than a “second portion.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

A hallux valgus deformity can cause soft tissue problems, such as pain and functional deficit. For example, a hallux valgus deformity can give rise to an impaired gait characterized by lateral and posterior weight shift, late heel rise, decreased single-limb balance, pronation deformity, and the like. When the hallux is deviating away from its normal position, a number of other problems may develop, including plantar fasciitis, shin splints, or other ankle or knee pathologies. Given that hallux valgus is relatively prevalent in the general population, there is an ongoing need for the development of foot treatment capabilities such as that related to, for example, treating hallux valgus deformities. Provided herein are embodiments and methods for manipulating the orientation of a 1^{st} metatarsal bone of a patient to correct the hallux valgus angle, as well as maintaining the orientation of the 1^{st} metatarsal bone during preparation of the 1^{st} metatarsocuneiform joint for arthrodesis.

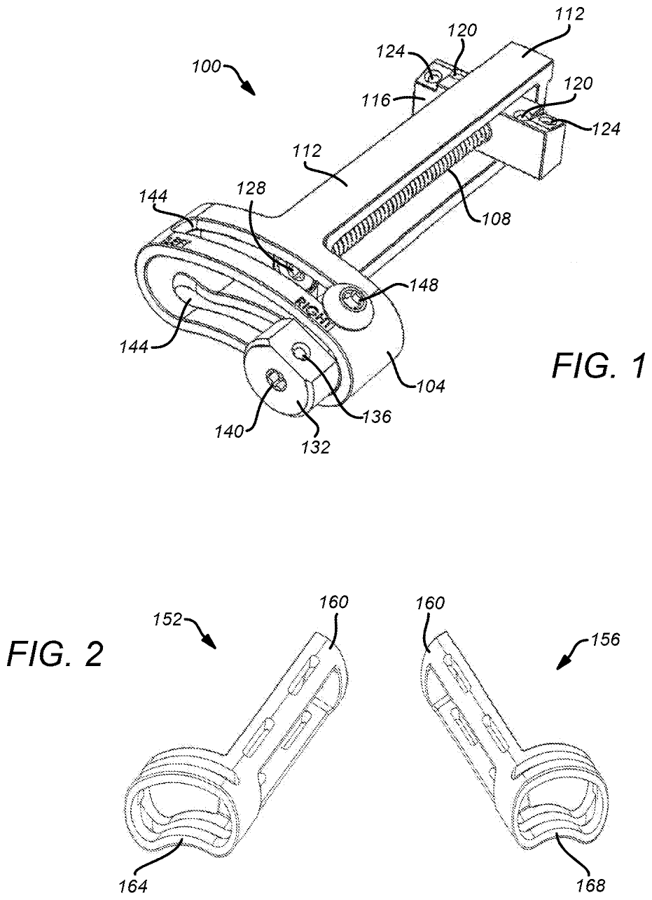

FIG. 1 illustrates an exemplary embodiment of a metatarsal alignment apparatus 100 configured for use on either a left foot or a right foot of a patient, according to the present disclosure. The apparatus 100 is configured to enable a surgeon to re-align, or orient, the 1^{st} metatarsal bone of a patient and correct the hallux valgus angle, as well as maintain the orientation of the 1^{st} metatarsal bone during preparation of the 1^{st} metatarsocuneiform joint for arthrodesis.

The metatarsal alignment apparatus 100 includes a distal frame 104 configured for use on the left foot or right foot of the patient. A threaded shaft 108 is longitudinally disposed within a proximal frame 112 and threadably engaged with a cuneiform block 116. The cuneiform block 116 includes vertical holes 120 and angled holes 124 suitable for pinning the cuneiform block 116 to the cuneiform bone. The diameters of the holes 120, 124 may range between substantially 0.5 mm and substantially 3.0 mm, without limitation. The angle of the angled holes 124 with respect to vertical holes 120 may range between about 5° and about 20°, without limitation. The proximal frame 112 is configured to guide the cuneiform block 116 in a longitudinal direction as the threaded shaft 108 is turned. A shaped opening 128 disposed in a distal end of the threaded shaft 108 facilitates engaging a suitable rotary tool with the threaded shaft 108 for the purpose of turning the threaded shaft 108 to move the cuneiform bone.

As shown in FIG. 1, the distal frame 104 houses a metatarsal block 132 configured for manipulating the position of the patient's 1^{st} metatarsal bone, as described herein. The metatarsal block 132 includes a cannulation 136 configured for pinning the metatarsal block 132 to the 1^{st} metatarsal bone. The diameter of the cannulation 136 may range between about 0.5 mm and about 3.0 mm, without limitation. Further, the metatarsal block 132 includes a shaped opening 140 configured for receiving a suitable tool whereby the metatarsal block 132 may be moved along slots 144 disposed in the distal frame 104. As shown in FIG. 1, the slots 144 are configured to rotate the metatarsal block 132 with respect to the cuneiform block 116. A lock screw 148 may be tightened to fixate the orientation of the metatarsal block 132 with respect to the distal frame 104.

It should be understood that the threaded shaft 108 and the slots 144 may be used to translate and rotate the 1^{st} metatarsal bone with respect to the cuneiform bone. For example, the threaded shaft 108 may include right-hand threads or left-hand threads for respectively distracting or compressing the 1^{st} metatarsal and cuneiform bones, as desired. Further, the slots 144 facilitate translating the 1^{st} metatarsal bone along a transverse plane and rotating the 1^{st} metatarsal bone with respect to the cuneiform bone. The lock screw 148 can be used to lock the rotation angle of the 1^{st} metatarsal bone at an angle of +/−30° with respect to the cuneiform bone.

It is contemplated that although the metatarsal alignment apparatus 100 of FIG. 1 is configured for bilateral use on left feet and right feet; in some embodiments, the apparatus 100 may be configured strictly for use on either left feet or right feet. For example, FIG. 2 illustrates an exemplary embodiment of a left frame 152 and a right frame 156 that are respectively configured for implementation in a left-foot metatarsal alignment apparatus and a right-foot metatarsal alignment apparatus in accordance with the present disclosure. The left frame 152 and the right frame 156 each comprises a proximal portion 160 that is configured to support a threaded shaft 108 and guide a cuneiform block 116 in a longitudinal direction, as described with respect to FIG. 1. The left frame 152 comprises a distal portion 164 that is configured to transversely translate and rotate a 1^{st} metatarsal bone with respect to a cuneiform bone of the left foot. Meanwhile, the right frame 156 comprises a distal portion 168 that is configured to transversely translate and rotate a 1^{st} metatarsal bone with respect to a cuneiform bone of the right foot.

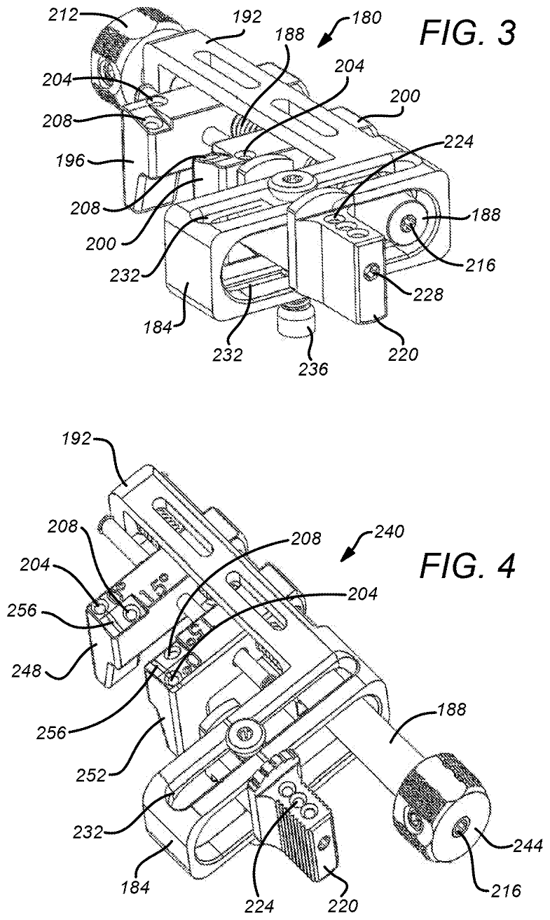

FIG. 3 illustrates an exemplary embodiment of a metatarsal alignment apparatus 180 configured for use on a left foot of a patient, according to the present disclosure. The apparatus 180 is configured to enable a surgeon to orient the 1^{st} metatarsal bone of a patient and correct the hallux valgus angle, as well as to fixate the orientation of the 1^{st} metatarsal bone during preparation of the 1^{st} metatarsocuneiform joint for arthrodesis.

The apparatus 180 includes a distal frame 184 configured for use on the left foot of the patient. A threaded shaft 188 is longitudinally disposed within a proximal frame 192 and threadably engaged with a cuneiform block 196 and a metatarsal block 200. The cuneiform block 196 includes vertical holes 204 and angled holes 208 suitable for pinning the cuneiform block 196 to the cuneiform bone. Similarly, the metatarsal block 200 includes vertical holes 204 and angled holes 208 for pinning the metatarsal block 200 to the 1^{st} metatarsal bone. The diameters of the holes 120, 124 generally ranges between about 0.5 mm and about 3.0 mm, without limitation. The angled holes 124 may be angled at between about 5° and about 20° with respect to the vertical holes 120, without limitation.

The proximal frame 192 is configured to guide the cuneiform block 196 and the metatarsal block 200 along the longitudinal direction as the threaded shaft 188 is turned. The threaded shaft 188 engages the cuneiform block 196 with left-hand threads and engages the metatarsal block 200 with right-hand threads. Thus, turning the threaded shaft 188 causes the cuneiform block 116 and the metatarsal block 200 to move in opposite directions. For example, in one embodiment, turning a knob 212 clockwise to rotate the threaded shaft 188 causes the blocks 196, 200 to move away from one another. As such, turning the knob 212 clockwise may be used to distract the 1^{st} metatarsal bone and the cuneiform bone during treating the hallux valgus angle of the left foot. Further, as shown in FIG. 3, a shaped opening 216 is disposed in a distal end of the threaded shaft 188. The shaped opening 216 is configured to facilitate engaging a suitable rotary tool with the threaded shaft 188 for the purpose of turning the threaded shaft 188 to distract or compress the 1^{st} metatarsal and cuneiform bones.

With continuing reference to FIG. 3, the distal frame 184 houses a distal metatarsal block 220 configured for manipulating the orientation of the patient's 1^{st} metatarsal bone, as described herein. The distal metatarsal block 220 includes vertical holes 224 configured for pinning the distal metatarsal block 220 to the 1^{st} metatarsal bone of the patient. The diameter of the vertical holes 224 may range between about 0.5 mm and about 3.0 mm, without limitation. Further, the distal metatarsal block 220 includes a shaped opening 228 configured for receiving a suitable tool whereby the block 220 may be moved along slots 232 disposed in the distal frame 184. The slots 232 are configured to allow the distal metatarsal block 220 to move along a transverse direction with respect to the proximal frame 192 as well as allow the distal metatarsal block 220 to rotate about its axis with respect to the cuneiform block 196. A distal head lock screw 236 may be tightened to fixate the orientation of the distal metatarsal block 220 with respect to the distal frame 184. It should be understood, therefore, that the slots 232 and the distal metatarsal block 220 facilitate translating the 1^{st} metatarsal bone along a transverse plane and rotating the 1^{st} metatarsal bone with respect to the cuneiform bone. The distal head lock screw 236 can be used to lock the position and rotation angle of the 1^{st} metatarsal bone with respect to the cuneiform bone.

FIG. 4 illustrates an exemplary embodiment of a metatarsal alignment apparatus 240 configured for use on a left foot of a patient, according to the present disclosure. The apparatus 240 is configured to enable a surgeon to orient the 1^{st} metatarsal bone of the patient and correct the hallux valgus angle, as well as maintain the orientation of the 1^{st} metatarsal bone during preparation of the 1^{st} metatarsocuneiform joint for arthrodesis.

The metatarsal alignment apparatus 240 shown in FIG. 4 is substantially similar to the metatarsal alignment apparatus 180, shown in FIG. 3, with the exception that the metatarsal alignment apparatus 240 includes a distal knob 244 that is coupled to a distal end of the threaded shaft 188, in lieu of the knob 212 shown in FIG. 3. Further, the metatarsal alignment apparatus 240 includes a cuneiform block 248 and a metatarsal block 252 that are threadably engaged with the shaft 188, such that clockwise rotation of the distal knob 244 causes the cuneiform block 248 and the metatarsal block 252 to move away from one another. As such, turning the distal knob 244 clockwise may be used to distract the cuneiform and 1^{st} metatarsal bones during treating a hallux valgus deformity.

As shown in FIG. 4, the cuneiform block 248 includes vertical holes 204 and angled holes 208 suitable for pinning the cuneiform block 248 to the cuneiform bone. Similarly, the metatarsal block 252 includes vertical holes 204 and angled holes 208 for pinning the metatarsal block 252 to the 1^{st} metatarsal bone. As described hereinabove, the diameter of the holes 120, 124 generally ranges between about 0.5 mm and about 3.0 mm, without limitation. The angled holes 124 may be angled between about 5° and about 20° with respect to the vertical holes 120, without limitation. Further, the vertical holes 204 may be disposed near a medial edge of the cuneiform block 248 and the metatarsal block 252 to facilitate pining along a center of the longitudinal axis of the 1^{st} metatarsal bone. As shown in FIG. 4, the cuneiform block 248 and the metatarsal block 252 may include alignment lines 256 to indicate the center of the longitudinal axis of the 1^{st} metatarsal bone. As such, the alignment lines 256 facilitate aligning the 1^{st} metatarsal bone and angling the distal head of the 1^{st} metatarsal with respect to the cuneiform bone.

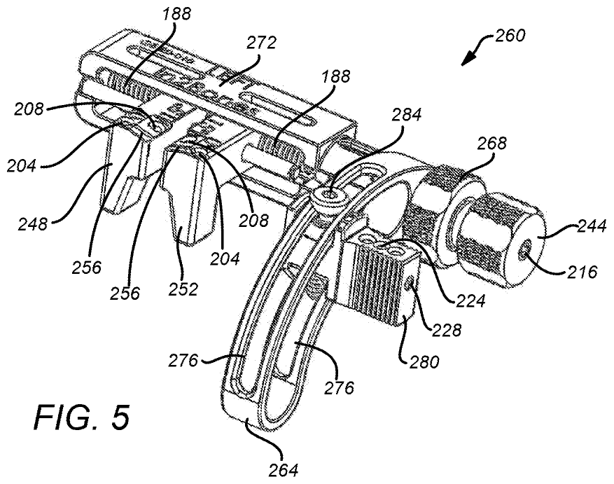

FIG. 5 illustrates an exemplary embodiment of a metatarsal alignment apparatus 260 configured for use on a left foot of a patient, according to the present disclosure. The apparatus 260 is configured to enable a surgeon to orient the 1^{st} metatarsal bone of the patient and correct the hallux valgus angle, as well as maintain the orientation of the 1^{st} metatarsal bone during preparation of the 1^{st} metatarsocuneiform joint for arthrodesis.

The metatarsal alignment apparatus 260 shown in FIG. 5 is similar to the metatarsal alignment apparatus 240 of FIG. 4, with an exception that the metatarsal alignment apparatus 260 includes an independent distal frame 264 and a locking knob 268 for fixating the distal frame 264 with respect to a proximal frame 272. It is contemplated that the distal frame 264 may be used to adjust the rotation angle of the 1^{st} metatarsal bone with respect to the cuneiform bone as well as enable a surgeon to rotate the distal frame 264 away for better access and visibility of the metatarsocuneiform joint. As will be appreciated, the surgeon may tighten the locking knob 268 to fixate the distal frame 264 with respect to the proximal frame 272.

Similar to the metatarsal alignment apparatus 240 of FIG. 4, the apparatus 260 illustrated in FIG. 5 includes a distal knob 244 that is coupled to a distal end of a threaded shaft 188. Further, the metatarsal alignment apparatus 260 includes a cuneiform block 248 and a metatarsal block 252 that are threadably engaged with the shaft 188, such that clockwise rotation of the distal knob 244 distracts the cuneiform block 248 and the metatarsal block 252. As such, the distal knob 244 may be turned clockwise to distract the cuneiform and 1^{st} metatarsal bones during treating a hallux valgus deformity, as described herein. In some embodiments, the threaded shaft 188, the cuneiform block 248, and the metatarsal block 252 may be configured to compress the cuneiform and 1^{st} metatarsal bones during clockwise rotation of the distal knob 244, without limitation.

With continuing reference to FIG. 5, the cuneiform block 248 includes vertical holes 204 and angled holes 208 suitable for pinning the cuneiform block 248 to the cuneiform bone. Similarly, the metatarsal block 252 includes vertical holes 204 and angled holes 208 for pinning the metatarsal block 252 to the 1^{st} metatarsal bone. As described hereinabove, the diameter of the holes 120, 124 may range between about 0.5 mm and about 3.0 mm while the angled holes 124 may be angled between about 5° and about 20° with respect to the vertical holes 120, without limitation. Further, the vertical holes 204 may be disposed near a medial edge of the cuneiform block 248 and the metatarsal block 252 to facilitate pining along a center of the longitudinal axis of the 1^{st} metatarsal bone. The cuneiform block 248 and the metatarsal block 252 may include alignment lines 256, as shown in FIG. 5, to indicate the center of the longitudinal axis of the 1^{st} metatarsal bone. As such, the alignment lines 256 facilitate orienting the 1^{st} metatarsal bone and angling the distal head of the 1^{st} metatarsal with respect to the cuneiform bone.

Turning, again, to FIG. 5, the distal frame 264 supports a distal metatarsal block 280 configured for manipulating the orientation of the patient's 1^{st} metatarsal bone, as described herein. The distal metatarsal block 280 includes vertical holes 224 configured for pinning the distal metatarsal block 280 to the 1^{st} metatarsal bone. The diameter of the vertical holes 224 may range between about 0.5 mm and about 3.0 mm, without limitation. Further, the distal metatarsal block 280 includes a shaped opening 228 configured for receiving a suitable tool whereby the block 280 may be moved along slots 276 disposed in the distal frame 264. The slots 276 are configured to allow the distal metatarsal block 280 to move along a transverse direction with respect to the proximal frame 272 and allow the distal metatarsal block 280 to rotate about its axis with respect to the cuneiform block 248. A distal head lock screw 284 may be tightened to fixate the orientation of the distal metatarsal block 280 with respect to the distal frame 264. As such, it should be understood that the slots 276 and the distal metatarsal block 280 facilitate translating the 1^{st} metatarsal bone along a transverse plane and rotating the 1^{st} metatarsal bone with respect to the cuneiform bone. The distal head lock screw 284 can be used to lock the orientation and angle of the 1^{st} metatarsal bone with respect to the cuneiform bone.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. To the extent there are variations of the invention, which are within the spirit of the disclosure or equivalent to the inventions found in the claims, it is the intent that this patent will cover those variations as well. Therefore, the present disclosure is to be understood as not limited by the specific embodiments described herein, but only by scope of the appended claims.

What is claimed is:

1. A metatarsal alignment apparatus, comprising:
a cuneiform block slidably disposed within a proximal frame;
a distal metatarsal block adjustably disposed within a distal frame; and
a threaded shaft for moving the cuneiform block relative to the distal metatarsal block.

2. The apparatus of claim 1, wherein the distal frame is configured for use on either a left foot or a right foot of a patient.

3. The apparatus of claim 1, wherein the threaded shaft is longitudinally disposed within the proximal frame and threadably engaged with the cuneiform block.

4. The apparatus of claim 1, wherein the proximal frame is configured to guide the cuneiform block in a longitudinal direction as the threaded shaft is turned.

5. The apparatus of claim 1, wherein the threaded shaft includes a shaped opening configured to be engaged with a suitable rotary tool for the purpose of turning the threaded shaft to move a cuneiform bone.

6. The apparatus of claim 1, wherein the cuneiform block includes vertical holes and angled holes for pinning the cuneiform block to the cuneiform bone.

7. The apparatus of claim 1, wherein the distal metatarsal block includes a cannulation configured for pinning the distal metatarsal block to a 1^{st} metatarsal bone.

8. The apparatus of claim 7, wherein the distal metatarsal block includes a shaped opening for receiving a tool whereby the distal metatarsal block may be moved along slots disposed in the distal frame.

9. The apparatus of claim 8, wherein the slots are configured to rotate the distal metatarsal block with respect to the cuneiform block.

10. The apparatus of claim 9, wherein a lock screw is configured to be tightened to fixate the orientation of the distal metatarsal block with respect to the distal frame.

11. The apparatus of claim 1, wherein the threaded shaft is longitudinally disposed within the proximal frame and threadably engaged with both the cuneiform block and a metatarsal block.

12. The apparatus of claim 11, wherein the metatarsal block includes vertical holes and angled holes for pinning the metatarsal block to a 1^{st} metatarsal bone.

13. The apparatus of claim 11, wherein the proximal frame is configured to guide the cuneiform block and the metatarsal block along the longitudinal direction as the threaded shaft is turned.

14. The apparatus of claim 13, wherein the threaded shaft engages the cuneiform block with left-hand threads and engages the metatarsal block with right-hand threads, such that turning the threaded shaft causes the cuneiform block and the metatarsal block to move in opposite directions.

15. The apparatus of claim 1, wherein the distal metatarsal block includes vertical holes configured for pinning the distal metatarsal block to a 1^{st} metatarsal bone of the patient.

16. The apparatus of claim 1, wherein the distal metatarsal block includes a shaped opening configured for receiving a suitable tool whereby the block may be moved along slots disposed in the distal frame.

17. The apparatus of claim 16, wherein the slots are configured to allow the distal metatarsal block to move along a transverse direction with respect to the proximal frame and to rotate about its axis with respect to the cuneiform block.

18. The apparatus of claim 17, wherein a distal head lock screw is configured to be tightened to fixate the orientation of the distal metatarsal block with respect to the distal frame.

19. The apparatus of claim 1, wherein the distal frame is configured to be rotated with respect to the proximal frame and to be fixated to the proximal frame by way of a locking knob.

20. The apparatus of claim 19, wherein the distal frame is configured to be used to adjust a rotation angle of the 1^{st} metatarsal bone with respect to the cuneiform bone.
